(19) United States
(12) Patent Application Publication
(10) Pub. No.: US 2007/0110622 A1
(43) Pub. Date: May 17, 2007

(54) Apparatus for monitoring the concentration of a gas

(75) Inventor: Ernest Phillipus Van Eeden, Constantia Kloof (ZA)

Correspondence Address: Anthony A Laurentano;Lahive & Cockfield, 30th Floor, One Post Office Square, Boston, MA, 02109, US

(73) Assignee: Anglo Operations Limited, Johannesburg (ZA)

(21) Appl. No.: 10/572,271

(22) PCT Filed: Sep. 14, 2004
(86) PCT No.: PCT/IB04/02974

§ 371 Date: Jan. 19, 2007

(30) Foreign Application Priority Data
Sep. 15, 2003 (ZA) 2003/7206

(51) Int. Cl. G01N 33/00

(52) U.S. Cl. 422/83

(57) ABSTRACT

Apparatus for monitoring the concentration of gas includes a gas intake pipe (5) adapted to pass through a barrier (2) into a sealed off area (1) to sample the gas concentration in the sealed off area. A pump is connected to the gas intake pipe for pumping gas from the sealed off area to a gas analyzer, which in use is placed outside the sealed off area so that the gas within the sealed off area can be analyzed. An air return pipe (6) is adapted to pass through the barrier into the sealed off area to return gas which has been sampled back to the sealed off area.

### BACKGROUND OF THE INVENTION

This invention relates to apparatus for monitoring the concentration of a gas, particularly a methane gas.

It is often necessary in a mining operation to seal off areas of the mine where methane gas is released. In sealed-off areas, the methane gas concentration will increase through an explosive range until oxygen is replaced. During the explosive concentration period of methane, lightning may for instance ignite the gas.

To prevent an explosion from causing damage, thick barrier walls are erected. Furthermore, effective sealing of the barrier walls prevents methane gas entering the workings of the mine and prevents fresh air being drawn into the sealed-off area which would increase the explosive risk.

However, in these cases the pressure differential between inside and outside the sealed off area makes taking measurements from within the sealed off area difficult.

The present invention seeks to address this by providing an improved system for monitoring methane gas.

### SUMMARY OF THE INVENTION

According to the present invention there is provided apparatus for monitoring the concentration of gas, the apparatus comprising:
a gas intake pipe adapted to pass through a barrier into a sealed off area to sample the gas concentration in the sealed off area;
a gas analyzer;
a pump connected to the gas intake pipe for pumping gas from the sealed off area to the gas analyzer, which in use is placed outside the sealed off area so that the gas within the sealed off area can be analyzed; and
an air return pipe adapted to pass through the barrier into the sealed off area to return gas which has been sampled back to the sealed off area.

Preferably, the apparatus includes a differential pressure transducer for monitoring the pressure within the sealed off area.

Preferably, both the gas intake pipe and the gas return pipe have flame traps located therein.

The length of the gas intake pipe is preferably long enough so that the gas sampled is not gas which would be affected by the breathing of the air in the sealed off area.

The present invention extends to a method of sampling gas comprising the steps of:
locating a sampling device on the other side of a barrier which seals off an area within which the gas to be sampled is located;
passing a gas intake pipe through the barrier into the sealed off area to sample the gas concentration in the sealed off area;
passing an air return pipe through the barrier into the sealed off area to return gas which has been sampled back to the sealed off area;
sampling the gas from the sealed off area and returning the sampled gas to the sealed off area.

### BRIEF DESCRIPTION OF THE DRAWINGS

FIG. 1 is a schematic diagram illustrating the apparatus of the present invention in use; and

FIG. 2 is a block circuit diagram of the apparatus of the present invention.

### DESCRIPTION OF EMBODIMENTS

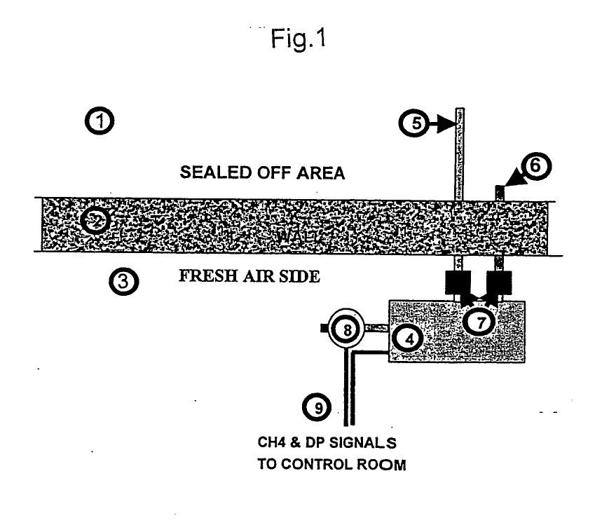

Referring to FIG. 1, a sealed-off area 1 contains methane gas concentration varying between 0% to 100% of methane gas. The area is sealed off using a barrier 2, typically a concrete barrierwall.

The fresh air side 3 is the side which is normally the operational side of the mine working.

With large variations in pressure between the sealed air and the fresh air side 3, conventional monitoring of gas sampling in the sealed area creates problems.

The variation in pressure may be up to 700 pa. A suitable sampling pump must be used to overcome the maximum differential vacuum. However, when barometric conditions change, the flow increases and a flow regulator needs to be used to reduce and control the sampling gas flow rate.

Loading on the sampling pump reduces its life. In addition, the flow rate must be kept high to maintain the pump's efficiency but high flow rates may clog the filter rapidly.

A high differential vacuum across the sampling pump would cause condensation affecting the monitoring devices and the sampling pump.

The present invention seeks to address this by being configured to achieve a balanced pressure.

To address this, a gas intake pipe 5 is adapted to pass through a barrier into a sealed off area to sample the gas concentration in the sealed off area. The gas intake pipe is adapted in that it has extra length and in the prototype took the form of a 30 metre sampling hose 5 which extends through or under the concrete barrier wall into the closed off area. The sampling hose 5 is used as a gas intake into the sampling device.

The sampling hose 5 extends quite far into the sealed off area as breathing through the concrete barrier may affect the gas concentration closer to the wall.

Similarly, an air return pipe is adapted to pass through the barrier into the sealed off area to return gas which has been sampled back to the sealed off area. The air return pipe is adapted in that it has extra length and in the prototype took the form of a sampling return hose 6 which is also inserted through or under the barrier wall 2 and protrudes just through the wall.

The fact that the sampled gas is returned to the sealed off area means that varying pressure differentials between the input sampling hose 5 and the return hose 6 are minimized.

The apparatus of the present invention is housed in a flameproof enclosure which houses the methane gas analyzer and sampling pump.

The device also includes a flame trap 7 installed in both sampling lines.

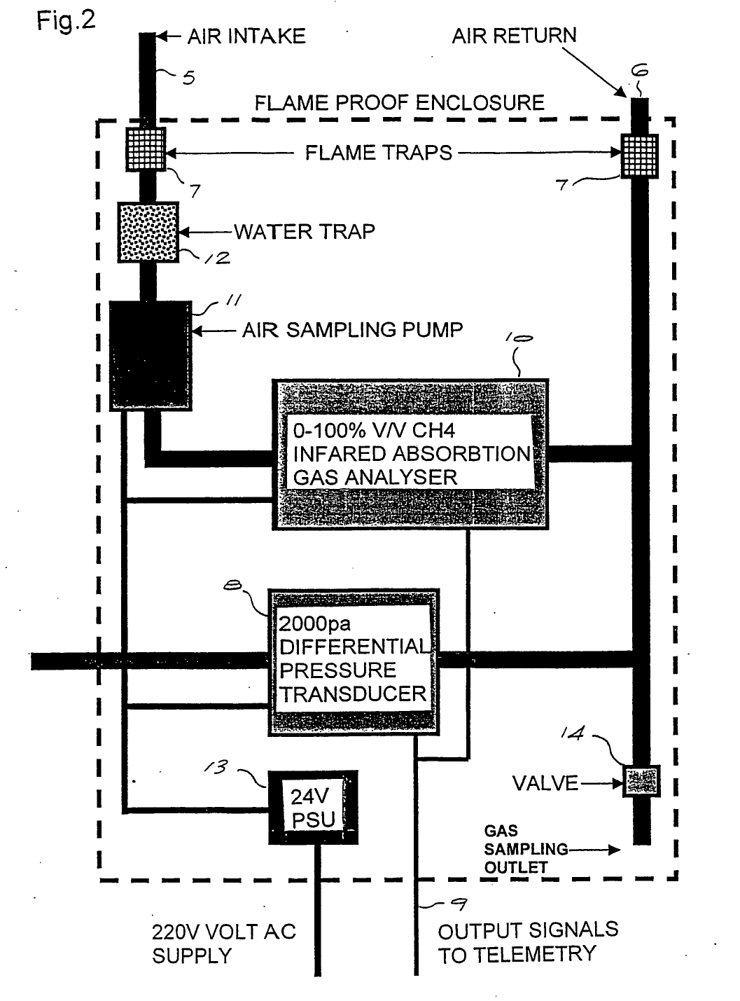

Referring to FIG. 2, the figure illustrates a block circuit diagram of the apparatus of the present invention.

A sample of the gas in the sealed off area is drawn through the air intake sampling hose 5. An air sampling pump 11 draws the air into the enclosure through the flame traps 7 and a water trap 12. The flame traps 7 and water trap 12 are well known in the art and will not be described here in detail. The flame traps typically comprise of a free copper mesh and the function of the flame traps 7 is to prevent fire as a result of an electrical fault in the flameproof enclosure penetrating the sealed off area.

The function of the water trap 12 is to keep water vapour off the instrumentation thereby to prevent condensation on the infrared measurement lenses.

An infrared absorption gas analyzer 10 analyses the sample to determine the concentration of methane. The gas analyzer is a typical gas analyzer such as a GFG™ analyzer. It will be appreciated that any other suitable gas analyzer could equally be used.

The device also includes a differential pressure transducer 8. The differential pressure transducer 8 monitors the differential pressure of the sealed off area 1 and compares it with the barometric pressure which indicates the effect of the ceiling of the walls and the monitoring equipment.

The differential pressure transducer 8 is well known in the art and will not be described in detail.

A power supply unit 13 supplies power to the various components of the apparatus.

A valve 14 is located between the apparatus and a gas sampling outlet. The function of the valve is to allow a sample of the measured gas to be taken to a laboratory for measurement to ensure that the device is accurately analyzing gas samples.

Once the measurements have been taken, the measurements are conveyed to the surface control room using telemetry equipment and signal lines 9.

The equipment of the present invention can be installed without affecting the wall structure and can be safely maintained as it lies on the operational side of the concrete barrier.

The equipment is not influenced by pressure changes between the in and out side of the sealed off area. These can be quite significant with the pressure differential reaching negative and positive values of up to 700 pA. It is the large negative pressure differential which makes the sampling of gas difficult in prior art systems and increases the risk of fresh air entering the sealed area.

The monitoring of the differential pressure off the sealed off area and comparing it with the barometric pressure will indicate the effectiveness of the sealing of the walls and the monitoring equipment

Furthermore, electrical faults on the equipment will not ignite the gas in the sealed off area due to the installation of flame traps 7 in the sampling lines.

With the gas monitoring apparatus configured to achieve a balanced pressure, a smaller sampling pump can be used. The flow rate will only be affected by the flow resistance of the sampling tubes and will be constant as there will be no differential pressure changes due to the fact that the sampling intake and outlet occur at the same pressure. Thus, sampling through the methane detector can be done more effectively as with no differential pressure changes a low and constant flow rate 10 is maintained.

The differential pressure transducer enables maintenance work to be done when the pressure is neutral and also to have an indication and alarm in the event of a seal failure.

1. Apparatus for monitoring the concentration of gas, the apparatus comprising:
a gas intake pipe adapted to pass through a barrier into a sealed off area to sample the gas concentration in the sealed off area;
a pump connected to the gas intake pipe for pumping gas from the sealed off area to the apparatus, which in use is placed outside the sealed off area so that the gas within the sealed off area can be analyzed;
a gas return pipe adapted to pass through the barrier into the sealed off area to return gas which has been sampled back to the sealed off area; and
a gas analyzer connected between the gas intake pipe and the gas return pipe so that pressure differentials between these pipes are minimized.

2. Apparatus according to claim 1 further comprising a differential pressure transducer for monitoring the pressure within the sealed off area.

3. Apparatus according to claim 1 wherein both the gas intake pipe and the gas return pipe have flame traps located therein.

4. Apparatus according to claim 1 wherein a length of the gas intake pipe is sufficiently long enough so that the gas sampled is not gas which would be affected by the breathing of air in the sealed off area.

5. (canceled)

6. A method of samples gas comprising the steps of:
passing a gas intake pipe through a barrier into a sealed off area to sample a gas concentration in the sealed off area;
passing a gas return pipe through the barrier into the sealed off area to return gas which has been sampled back to the sealed off area;
locating a sampling device on the other side of a barrier which seals off an area within which the gas to be sampled is located, the sampling device being connected between the gas intake pipe and the gas return pipe so that pressure differentials between these pipes are minimized; and
sampling the gas from the sealed off area and returning the sampled gas to the sealed off area.
